(19) United States
(12) Patent Application Publication
(10) Pub. No.: US 2019/0054144 A1
(43) Pub. Date: Feb. 21, 2019

(54) PHARMACEUTICAL COMPOSITION FOR TREATING ISCHEMIC STROKE AND METHOD THEREOF

(71) Applicant: Meridigen Biotech Co., Ltd., Taipei City (TW)

(72) Inventors: Chang-Yo HSUAN, Taipei City (TW); Willie LIN, Taipei City (TW); Yu-Chin SU, Taipei City (TW); Tang-bo Chung WU, Taipei City (TW)

(73) Assignee: Meridigen Biotech Co., Ltd., Taipei City (TW)

(21) Appl. No.: 15/677,352

(22) Filed: Aug. 15, 2017

(51) Int. Cl. A61K 38/18; A61K 35/28; A61K 38/38

(52) U.S. Cl. CPC A61K 38/1833; A61K 38/385; A61K 35/28

(57) ABSTRACT

Disclosed is a pharmaceutical composition for treating ischemic stroke, comprising an effective amount of human mesenchymal stem cells, human serum albumin, and a pharmaceutically acceptable carrier or diluent. Also disclosed is a method for treating ischemic stroke in a subject in need thereof, the method comprising administering to the subject a pharmaceutical composition comprising an effective amount of human mesenchymal stem cells, human serum albumin, and a pharmaceutically acceptable carrier or diluent. In addition, a method for promoting secretion of hepatocyte growth factor by human mesenchymal stem cells is disclosed.

### FIELD OF THE INVENTION

The present invention pertains to a pharmaceutical composition for treating ischemic stroke, as well as a method thereof. The present invention also relates to a method for promoting secretion of hepatocyte growth factor by human mesenchymal stem cells.

### BACKGROUND OF THE INVENTION

Recently, stem cells have become a promising candidate for cell-based therapy because of their multi-potency such as anti-apoptosis, anti-oxidative stress, angiogenesis and immuno-modulation [1-3]. Stem cells show huge potential in the treatment of many ischemic diseases including peripheral vascular diseases, stroke, and coronary artery diseases [4-6]. To date, many stem cells based clinical trials are held in ischemic stroke and already shown safety and efficacy.

The therapeutic effects of stem cells transplantation in ischemic stroke are mediated by the secretion of paracrine bioactive factors [7-8], which involve vascular endothelial growth factor (VEGF), hepatocyte growth factor (HGF), monocyte chemotactic protein-1 (MCP-1), interleukin-6 (IL-6), and transforming growth factor-beta1 (TGF-β1) [9-11]. Among these growth factors, HGF plays an important role in anti-oxidative stress and stroke treatment. HGF shows anti-oxidative stress capability by mediating MAPK/P38 pathway and reducing cell apoptosis [12-13]. Further, several studies revealed that stem cells showed beneficial effect in stroke treatment by secreting paracrine bioactive factors and improving behavior recovery [14-16].

U.S. Pat. No. 9,415,036 B2 discloses a pharmaceutical composition for the acute and/or chronic treatment or prevention of osteoarticular diseases including an adequate pharmaceutical carrier or diluent, a polysaccharide and/or a glycosaminoglycan, an anti-inflammatory agent and stem cells.

### BRIEF SUMMARY OF THE INVENTION

In one aspect, the present invention provides a pharmaceutical composition for treating ischemic stroke. The pharmaceutical composition includes an effective amount of human mesenchymal stem cells, human serum albumin, and a pharmaceutically acceptable carrier or diluent.

In another aspect, the present invention provides a method for treating ischemic stroke in a subject in need thereof. The method comprises the step of administering to the subject a pharmaceutical composition comprising an effective amount of human mesenchymal stem cells, human serum albumin, and a pharmaceutically acceptable carrier or diluent.

According to the present invention, the pharmaceutical composition may be prepared by a method comprising: mixing the human mesenchymal stem cells with the pharmaceutically acceptable carrier or diluent, which is supplemented with the human serum albumin, to allow the human serum albumin to promote the secretion of hepatocyte growth factor by the human mesenchymal stem cells.

Preferably, the human serum albumin is in an amount effective to promote the secretion of hepatocyte growth factor by the human mesenchymal stem cells in the pharmaceutical composition.

In a further aspect, provided is a method for promoting secretion of hepatocyte growth factor by human mesenchymal stem cells. The method comprises mixing the human mesenchymal stem cells with a pharmaceutically acceptable carrier or diluent containing human serum albumin in an amount effective to promote the secretion of hepatocyte growth factor by the human mesenchymal stem cells.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows that HGF is a critical factor in stem cell therapy for ischemic stroke.

FIG. 2 shows that HSA promotes secretion of HGF by mesenchymal stem cells.

FIG. 3A shows that HSA has no effects on viability of mesenchymal stem cells; and FIG. 3B shows that HSA has no effects on cell number of mesenchymal stem cells.

FIG. 4 shows that HSA enhances the efficacy of stem cell therapy for ischemic stroke.

FIG. 5 shows that the administration of HSA, HGF, or their combination without mesenchymal stem cells shows no significant therapeutic effects.

### DETAILED DESCRIPTION OF THE INVENTION

In one aspect, the present invention provides a pharmaceutical composition for treating ischemic stroke. The pharmaceutical composition includes an effective amount of human mesenchymal stem cells, human serum albumin, and a pharmaceutically acceptable carrier or diluent.

In another aspect, the present invention provides a method for treating ischemic stroke in a subject in need thereof. The method comprises the step of administering to the subject a pharmaceutical composition comprising an effective amount of human mesenchymal stem cells, human serum albumin, and a pharmaceutically acceptable carrier or diluent.

In certain preferred embodiments of the present invention, the human serum albumin presents in the pharmaceutical composition in an amount effective to promote the secretion of hepatocyte growth factor by the human mesenchymal stem cells.

The term “mesenchymal stem cells” as used herein includes cells isolated from tissues of adults, such as bone marrow, a fat cell, and a periodontal membrane, as well as cells isolated from tissues of fetus, placenta, and cord blood. In some examples of the present invention, the mesenchymal stem cells are derived from a placenta-related tissue selected from the group consisting of amniotic membrane, chorionic disk, chorionic membrane, and umbilical cord.

As used herein, the term “ischemic stroke” refers to a type of stroke that is of limited extent and caused by a blockage of blood flow. Ischemic stroke includes but is not limited to cerebral ischemia, stroke, ischemia after cardiac arrest, and complications from surgery.

According to one embodiment, the pharmaceutically acceptable carrier or diluent is a normal saline.

According to certain embodiments of the present invention, the amount of the human serum albumin is ranging from 0.5% (w/v) to 25% (w/v), preferably 1% (w/v) to 10% (w/v), based on the volume of the pharmaceutically acceptable carrier or diluent.

As used herein, w/v means g/mL.

According to the present invention, the pharmaceutical composition may be prepared by a process comprising: mixing the human mesenchymal stem cells with the pharmaceutically acceptable carrier or diluent, which is supplemented with the human serum albumin.

Said process may further comprise incubating the human mesenchymal stem cells, as a suspension culture, in the pharmaceutically acceptable carrier or diluent, for a period of time to allow the human serum albumin to promote the secretion of hepatocyte growth factor by the human mesenchymal stem cells. A person of ordinary skill in the art may determine, through routine experimentation, an adequate period of time for incubating the human mesenchymal stem cells, such that the human serum albumin reaches a desirable level in the pharmaceutical composition and that the pharmaceutical composition is more effective in treating ischemic stroke as compared to a pharmaceutical composition which is not “activated” by said process before use. In other words, the method for treating ischemic stroke of the present invention may further comprise the preliminary steps of: mixing the human mesenchymal stem cells with the pharmaceutically acceptable carrier or diluent, which is supplemented with the human serum albumin, and incubating the pharmaceutical composition to allow the human serum albumin to promote the secretion of hepatocyte growth factor by the human mesenchymal stem cells.

However, since a pharmaceutical composition comprising stem cells is generally administered to a subject in need thereof gradually over a relatively long period of time, the pharmaceutical composition of the present invention may also be used shortly after the human mesenchymal stem cells are mixed with the pharmaceutically acceptable carrier or diluent supplemented with human serum albumin. In such alternative embodiments, the secretion of hepatocyte growth factor by the human mesenchymal stem cells would be enhanced during the course of administration.

In a further aspect, provided is a method for promoting secretion of hepatocyte growth factor by human mesenchymal stem cells. The method comprises mixing the human mesenchymal stem cells with a pharmaceutically acceptable carrier or diluent containing human serum albumin in an amount effective to promote the secretion of hepatocyte growth factor by the human mesenchymal stem cells. As described above, such method may further comprise incubating the human mesenchymal stem cells, as a suspension culture, in the pharmaceutically acceptable carrier or diluent, for a period of time to allow the human serum albumin to promote the secretion of hepatocyte growth factor by the human mesenchymal stem cells. Similarly, a person of ordinary skill in the art may determine, through routine experimentation, an adequate period of time for incubating the human mesenchymal stem cells, such that the secretion of hepatocyte growth factor is effectively promoted.

The present invention is further illustrated by the following examples, which are provided for the purpose of demonstration rather than limitation.

### EXAMPLES

### Example 1: Preparation of Mesenchymal Stem Cells

Full-term placentas were collected after obtaining written informed consent from donors. MSCs were derived from amniotic membrane (AM), chorionic disk (CD), chorionic membrane (CM), and umbilical cord (UC). Placenta-derived mesenchymal stem cells were cultured, expanded and maintained in α-MEM with FBS and basic FGF at 37° C., saturating humidity and 5% CO₂, and were sub-cultured when cells reached 80% confluence. The cells are stored under low temperature conditions before use.

### Example 2: Hepatocyte Growth Factor (HGF) is a Critical Factor in Stem Cell Therapy for Ischemic Stroke

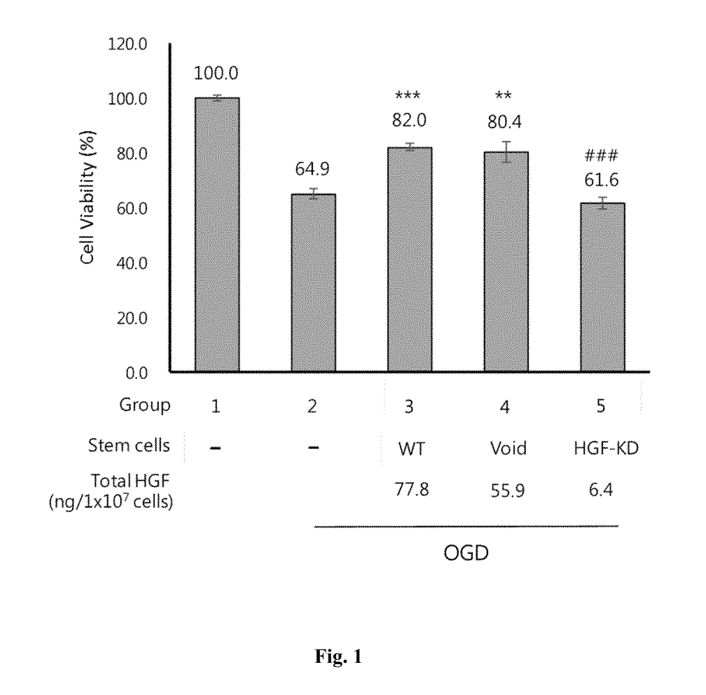

Human neuroblastoma (SH-SY5Y) cells cultured in 24-well plates were subjected to oxygen-glucose deprivation (OGD), mimic ischemic stroke conditions, for 4 hours. The OGD-damaged neuroblastoma cells were co-cultured with or without mesenchymal stem cells (prepared as described in Example 1) which are of wild type (WT), void control, and HGF knockdown (HGF-KD) for 24 hours. The mesenchymal stem cells were co-cultured with the OGD-damaged neuroblastoma cells by using 24-well insert plates with 0.4 μm membrane pore sizes. The void control and HGF-KD mesenchymal stem cells mesenchymal were prepared as follows: seed mesenchymal stem cells in 6 well plate (2×10⁵ cells/well) for 24 hours; add 1.5 μL of polybrene (8 μg/mL) with 1.5 mL complete medium for 10 minutes in each group; count cell number and drop void control/HGF-KD virus (MOI=10) in respective groups, then incubate overnight; passage cells and seed 6×10⁵ cells in T25 flask with 3 mL complete medium for 4 hours; add 3 mL complete medium with 6 μL of puromycin (1 μg/mL) and incubate for 48 hours; passage cells and seed 5.3×10⁵ cells in T75 flask with 10 mL complete medium and 10 μL of puromycin (1 μg/mL) for 3 days; after puromycin selection for 3 days, void control and HGF-KD mesenchymal stem cells were ready for use. Neuroblastoma cell viability was analyzed by MTT assay. HGF levels were measured by using HGF ELISA kit. The results are Shown in FIG. 1.

### Example 3: Human Serum Albumin (HSA) Promotes Secretion of HGF by Mesenchymal Stem Cells

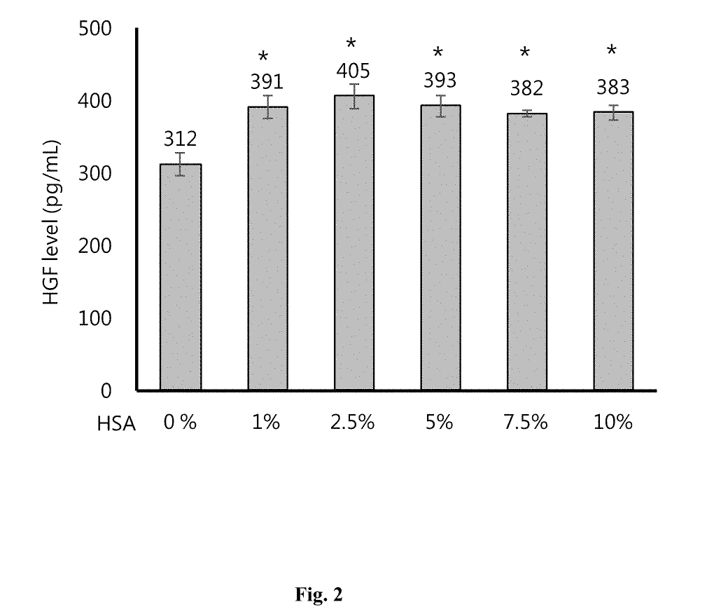

1×10⁷ mesenchymal stem cells (prepared as described in Example 1) were mixed with normal saline supplemented with different amounts (0, 1% (w/v), 2.5% (w/v), 5% (w/v), 7.5% (w/v), and 10% (w/v), based on the volume of normal saline) of HSA, and incubated for 4 hours. Subsequently, the mixture was subjected to centrifugation at 300×g for 5 minutes, the supernatant was collected and HGF level in the supernatant was measured by using HGF ELISA kit. The results are shown in FIG. 2.

### Example 4: HSA has No Effects on Viability and Cell Number of Mesenchymal Stem Cells

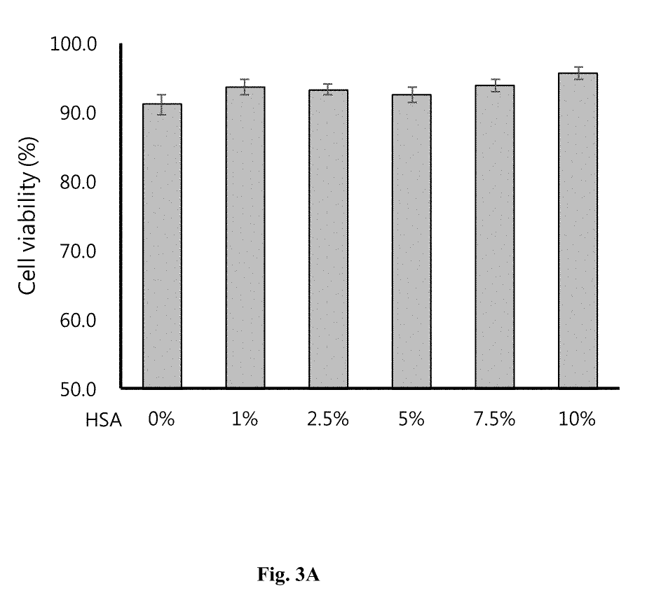

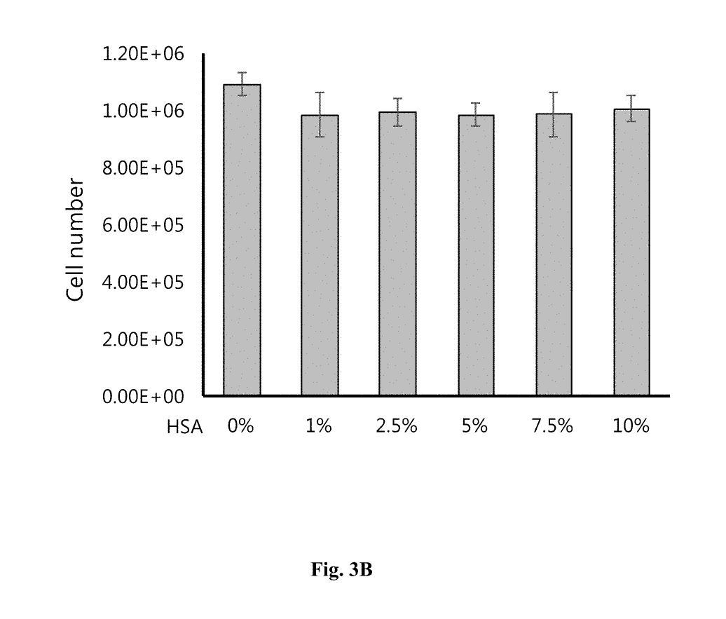

1×10⁷ mesenchymal stem cells (prepared as described in Example 1) were mixed with normal saline supplemented with different amounts (0, 1% (w/v), 2.5% (w/v), 5% (w/v), 7.5% (w/v), and 10% (w/v), based on the volume of normal saline) of HSA, and incubated for 4 hours. Cell viability and cell number were measured by a cell counter (NucleoCounter® NC-250, ChemoMetec). The results are shown in FIG. 3A and FIG. 3B.

### Example 5: HSA Enhances the Efficacy of Stem Cell Therapy for Ischemic Stroke

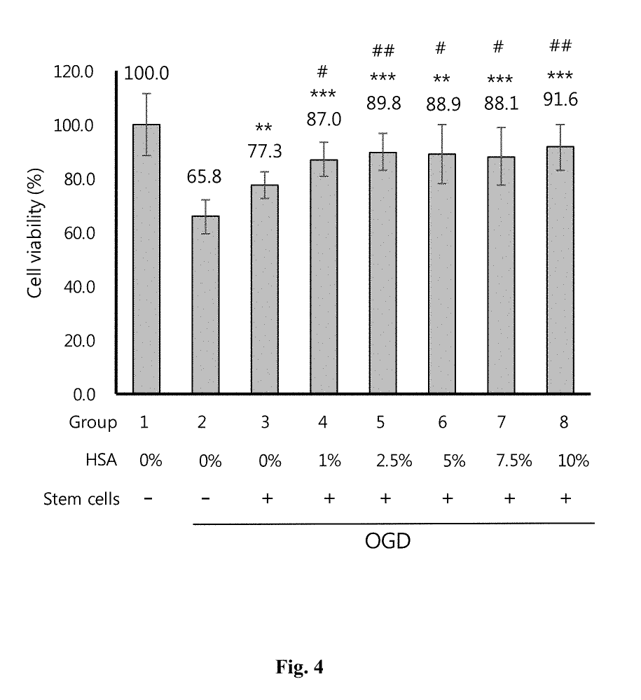

Human neuroblastoma (SH-SY5Y) cells cultured in 24-well plates were subjected to oxygen-glucose deprivation (OGD), mimic ischemic stroke conditions, for 4 hours. The OGD-damaged neuroblastoma cells were co-cultured with or without mesenchymal stem cells (prepared as described in Example 1) pre-incubated with different amounts of HSA as described in Example 4. The mesenchymal stem cells were co-cultured with the OGD-damaged neuroblastoma cells by using 24-well insert plates with 0.4 μm membrane pore sizes. Neuroblastoma cell viability was analyzed by MTT assay. The results are shown in FIG. 4.

### Example 6: Administration of HSA, HGF, or their Combination without Mesenchymal Stem Cells Shows No Significant Therapeutic Effects

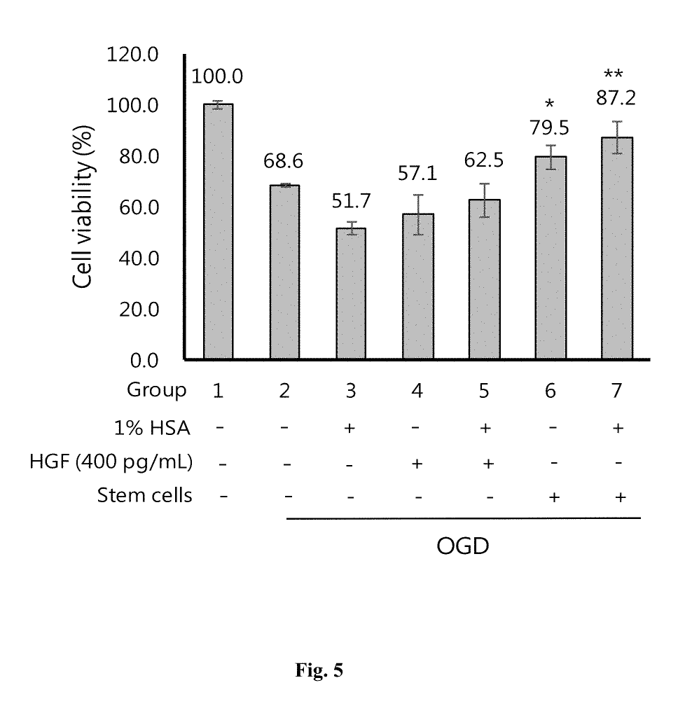

Human neuroblastoma (SH-SY5Y) cells cultured in 24-well plates were subjected to oxygen-glucose deprivation (OGD), mimic ischemic stroke conditions, for 4 hours. The OGD-damaged neuroblastoma cells were co-cultured with or without mesenchymal stem cells (prepared as described in Example 1) pre-incubated with different amounts of HSA as described in Example 4, or treated with 1% HSA, 400 pg/mL HGF, or 1% HSA plus 400 pg/mL HGF. The mesenchymal stem cells were co-cultured with the OGD-damaged neuroblastoma cells by using 24-well insert plates with 0.4 μm membrane pore sizes. Neuroblastoma cell viability was analyzed by MTT assay. The results are shown in FIG. 5.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCES

[1] Liao W, Xie J, Zhong J, Liu Y, Du L, Zhou B, Xu J, Liu P, Yang S, Wang J, Han Z, Han Z C. Therapeutic effect of human umbilical cord multipotent mesenchymal stromal cells in a rat model of stroke. Transplantation. 2009.
[2] Kim N, Cho S G. Clinical applications of mesenchymal stem cells. Korean J Intern Med. 2013.
[3] de Girolamo L, Lucarelli E, Alessandri G, Avanzini M A, Bernardo M E, Biagi E, Brini A T, D'Amico G, Fagioli F, Ferrero I, Locatelli F, Maccario R, Marazzi M, Parolini O, Pessina A, Torre M L, Italian Mesenchymal Stem Cell Group. Mesenchymal stem/stromal cells: a new “cells as drugs” paradigm. Efficacy and critical aspects in cell therapy. Curr Pharm Des. 2013.
[4] Wang S, Yu L, Sun M, Mu S, Wang C, Wang D, Yao Y. The therapeutic potential of umbilical cord mesenchymal stem cells in mice premature ovarian failure. Biomed Res Int. 2013.
[5] Misra V, Ritchie M M, Stone L L, Low W C, Janardhan V. Stem cell therapy in ischemic stroke: role of IV and intra-arterial therapy. Neurology. 2012.
[6] Tao H, Han Z, Han Z C, Li Z I. Proangiogenic Features of Mesenchymal Stem Cells and Their Therapeutic Applications. Stem Cells Int. 2016.
[7] Kwon H M, Hur S M, Park K Y, Kim C K, Kim Y M, Kim H S, Shin H C, Won M H, Ha K S, Kwon Y G, Lee D H I, Kim Y M. Multiple paracrine factors secreted by mesenchymal stem cells contribute to angiogenesis. Vascul Pharmacol. 2014.
[8] Drago D, Cossetti C, Iraci N, Gaude E, Musco G, Bachi A, Pluchino S. The stem cell secretome and its role in brain repair. Biochimie. 2013.
[9] Shen C, Lie P, Miao T, Yu M l, Lu Q, Feng Ti, Li J, Zu Ti, Liu X, Li H. Conditioned medium from umbilical cord mesenchymal stem cells induces migration and angiogenesis. Mol Med Rep. 2015.
[10] Pawitan J A. Prospect of stem cell conditioned medium in regenerative medicine. Biomed Res Int. 2014.
[11] Burchfield J S, Dimmeler S. Role of paracrine factors in stem and progenitor cell mediated cardiac repair and tissue fibrosis. Fibrogenesis Tissue Repair. 2008.
[12] Kunio M, Hiroshi F, Hisaaki T, Katsuya S. HGF-Met Pathway in Regeneration and Drug Discovery. Biomedicines. 2014.
[13] Simona G, Valentina S, Stefano G, Tiziana C. HGF/Met Axis in Heart Function and Cardioprotection. Biomedicines. 2014.
[14] Zhao M Z, Nonoguchi N, Ikeda N, Watanabe T, Furutama D, Miyazawa D, Funakoshi H, Kajimoto Y, Nakamura T, Dezawa M, Shibata M A, Otsuki Y, Coffin R S, Liu W D, Kuroiwa T, Miyatake S. Novel therapeutic strategy for stroke in rats by bone marrow stromal cells and ex vivo HGF gene transfer with HSV-1 vector. J Cereb Blood Flow Metab. 2006.
[15] Chen J, Shehadah A, Pal A, Zacharek A, Cui X, Cui Y, Roberts C, Lu M, Zeitlin A, Hariri R, Chopp M. Neuroprotective effect of human placenta-derived cell treatment of stroke in rats. Cell Transplant. 2013.
[16] Chen J, Venkat P, Zacharek A, Chopp M. Neurorestorative therapy for stroke. Front Hum Neurosci. 2014

What is claimed is:

1. A pharmaceutical composition for treating ischemic stroke, comprising:
an effective amount of human mesenchymal stem cells;
human serum albumin; and
a pharmaceutically acceptable carrier or diluent.

2. The pharmaceutical composition of claim 1, wherein the human serum albumin is in an amount effective to promote the secretion of hepatocyte growth factor by the human mesenchymal stem cells in the pharmaceutical composition.

3. The pharmaceutical composition of claim 1, which is prepared by a method comprising mixing the human mesenchymal stem cells with the pharmaceutically acceptable carrier or diluent, which is supplemented with the human serum albumin.

4. A method for treating ischemic stroke in a subject in need thereof, comprising:
administering to the subject a pharmaceutical composition comprising: an effective amount of human mesenchymal stem cells; human serum albumin; and a pharmaceutically acceptable carrier or diluent.

5. The method of claim 4, wherein the human serum albumin is in an amount effective to promote the secretion of hepatocyte growth factor by the human mesenchymal stem cells in the pharmaceutical composition.

6. A method for promoting secretion of hepatocyte growth factor by human mesenchymal stem cells, comprising:
mixing the human mesenchymal stem cells with a pharmaceutically acceptable carrier or diluent containing human serum albumin in an amount effective to promote the secretion of hepatocyte growth factor by the human mesenchymal stem cells.
